(19) United States
(12) Patent Application Publication
(10) Pub. No.: US 2024/0122667 A1
(43) Pub. Date: Apr. 18, 2024

(54) MEDICAL DEVICE STERILE BARRIER PACKAGING SYSTEM

(71) Applicant: GYRUS MEDICAL LIMITED, Cardiff (GB)

(72) Inventors: Rhodri JAMES, Cardiff (GB); Teo Heng Jimmy YANG, Cardiff (GB); Anna PRITCHARD, Cardiff (GB); Ryan VINCENT, Cardiff (GB); Josh WINN, Cardiff (GB)

(73) Assignee: GYRUS MEDICAL LIMITED, Cardiff (GB)

(21) Appl. No.: 18/379,465

(22) Filed: Oct. 12, 2023

Related U.S. Application Data
(60) Provisional application No. 63/416,165, filed on Oct. 14, 2022.

(51) Int. Cl. A61B 50/31; A61B 50/22; B65B 5/04; B65B 7/02; B65B 43/26; B65B 51/00; B65B 55/02; B65D 33/02; B65D 75/04; B65D 75/38; B65D 75/58; B65D 77/04

(52) U.S. Cl. CPC A61B 50/31; A61B 50/22; B65B 5/045; B65B 7/02; B65B 43/26; B65B 51/00; B65B 55/02; B65D 33/02; B65D 75/04; B65D 75/38; B65D 75/5805; B65D 77/04; A61B 2050/314

(57) ABSTRACT

A medical device sterile barrier system that is easier for surgical personnel to use by allowing easier opening and removal of the medical device from the sterile packaging, includes a sterile bag within which a medical device is held secure via a removable holster, the holster being fashioned with a hinge feature such that the holster has a clamshell arrangement and folds in half along the hinge feature to clamp the medical device parts in place between the two halves of the clamshell. To remove the medical device the holster is simply unfolded about the hinge feature to reveal the parts of the device, which can then be lifted out of the holster and assembled in the usual way. There are thus no polymer tab features forming part of the holster and interacting with the device to secure it in place that need removal before the device can be used.

### TECHNICAL FIELD

Embodiments of the present invention relate to a medical device sterile barrier packaging system, and in particular to such a system that improves usability and/or reduces waste materials with respect to the prior art.

### BACKGROUND TO THE INVENTION AND PRIOR ART

The packaging of medical devices, and particularly surgical devices that must be kept sterile, is subject to international standardization, under ISO 11607-1. This standard specifies the requirements for materials, sterile barrier systems and packaging systems for terminally sterilized medical devices. Annex D of the standard considers environmental aspects of sterile packaging for medical devices, with the objective of encouraging packaging design that minimizes environmental impact.

FIGS. 7 and 8 are images showing typical existing surgical device packaging. FIG. 7 in particular shows the very commonly used thermoform blister pack type of packaging. This type of packaging typically comprises a thermoformed PETG plastic tray 72, having separate thermoformed shaped compartments specifically shaped to contain the various parts of the medical device. The tray 72 (the underside of which is shown in FIG. 7), is sealed on its top surface with a removable adhesive sheet, which is typically formed from a non-woven breathable yet water-resistant material, such as the well known material Tyvek® available from DuPont®. In use the whole tray is brought in a sealed condition into the operating theatre, and then opened and presented aseptically to the surgeon or her assistant, who then removes the instruments from the thermoformed plastic tray (see left hand figure of FIG. 9).

One issue with the commonly used PETG thermoformed blister pack type of packaging is that it uses relatively a lot of plastic to accomplish the aseptic packaging, and results in a bulky package that is harder to transport and store. Moreover, the resulting waste packaging once the instrument has been used is also bulky and relatively costly to dispose of.

To help ameliorate these issues more recently surgical and other medical devices have begun to be supplied in envelope type packaging supported by cardboard die-cut support frames. One such example is shown in FIG. 8, which illustrates a LigaSure™ type dissector available from the medical devices company Covidien®.

As will be seen, the surgical dissector is supplied in a peel pouch 82 formed from Tyvek on one side and a clear plastic sheet on the other, with the instrument sandwiched in between. To keep the instrument in place within the bag, and particularly to provide a shield for the instrument end effectors (which can be sharp), the instrument is held within a polymer die cut frame 84. The frame 84 has a folded shield portion 88 into which the forceps jaws of the instrument are placed, the shield acting to prevent the forceps jaws from coming into contact with the inner surfaces of the bag 82. The frame 84 then extends the length of the instrument and the handles of the instrument are secured in place to the frame via polymer tabs 86, that are die cut from the frame and then folded over the instrument handles. In use, such an instrument is carried into the operating theater by a theater assistant, who then peels open one end of the envelope and aseptically presents the sterile instrument to the surgeon or his assistant, who then removes the instrument from the bag (see right hand figure of FIG. 9). However, at this point the instrument is still attached to the card frame, from which the instrument must then be removed for use. This can be a fiddly task, requiring the removal of the polymer tabs 86 from the instrument, and the withdrawal of the instrument from the shield 88.

Thus, whilst the arrangement of FIG. 8 helps to address the waste packaging issue by doing away with the thermoformed blister pack and replacing it with a much smaller envelope arrangement, it introduces some additional complication into the surgical procedure, as the surgeon must remove the instrument from the polymer frame and then discard the frame once the instrument has been removed from the aseptic packaging. In contrast, with the blister pack arrangement the instrument is available for immediate use once removed from the blister pack.

FIGS. 10 and 11 illustrate a known hybrid arrangement between the two arrangements above. Here, as shown in FIG. 10, various different types of medical instrument can be held in polymer die cut frames 102 of various design. Then, as shown in FIG. 11, the polymer die cut frames 102 containing the medical instruments can be packaged in a generic PETG thermoformed blister pack 104, which does not possess the instrument specific shaped compartments such as those contained in the tray 72 of FIG. 7. Instead, the device is held within the generic blister pack 104 via the polymer die cut frame 102. Such an arrangement has the disadvantages of both previously described arrangements, in that there is much bulky waste packaging in the form of the generic blister pack 104, plus once the instrument is removed from the sterile blister pack, the surgeon or his assistant is then presented with the fiddly task of removing the instrument from the polymer frame 102, by removal of the various polymer tabs holding the instrument or device in place on the frame.

There is thus still a need for a medical device sterile barrier packaging system that has a design that minimizes environment impact via the use of less material, or by using more environmentally friendly materials, but that is easier for surgical personnel to use during actual surgical procedures.

### SUMMARY OF THE INVENTION

Embodiments of the present disclosure present a medical device sterile barrier system that is easier for surgical personnel to use by allowing easier opening and removal of the medical device from the sterile packaging. In one embodiment the system includes a sterile bag within which a medical device is held secure via a removable holster, the holster being fashioned in one embodiment with a hinge feature such that the holster has a clamshell arrangement and folds in half along the hinge feature to clamp the medical device parts in place between the two halves of the clamshell. To remove the medical device the holster is simply unfolded about the hinge feature to reveal the parts of the medical device, which can then simply be lifted out of the holster and assembled in the usual way. There are thus no polymer tab features forming part of the holster and interacting with the medical device to secure it in place that need removal before the device can be used.

In the same or another embodiment the sterile bag is further provided with a “tear-off” or “tear open” seal at the top to facilitate easier opening of the bag. The bag may be of a shape that permits it to have a flat bottom so that it can stand upright, with flexible upstanding walls that are brought together and sealed together at the top to give a substantially triangular cross-section of bag. The seal is of a type that the parts of the bag that are brought together to form the seal can be torn off to allow the inside of the bag to be accessed, below the seal. This is easier to use than the prior art arrangements which require the bag or container to be peeled open, as the tearing off of the top of the bag is a more vigorous action that can be performed more quickly and with more certainty.

In one embodiment the interior of the bag is also provided with an internal frame to help the bag to keep its shape. The frame may be part of or separate to the holster within which the medical device parts are kept. The frame is preferably made of plastic or cardboard, and in use keeps the inner walls of the bag away from the medical device. In this respect, depending on the precise device, the medical device may have sharp edges or points that could penetrate the inner surface of the wall of the sterile bag, thus meaning that the inside of the bag and the medical device inside is no longer sterile. Providing the internal frame to keep the walls of the bag away from the device will prevent such penetration from occurring.

In view of the above, from a first aspect there is provided a medical device sterile barrier system, comprising: a flexible walled bag, the interior of which is sterile when in use; and a medical device holster contained within the bag, the medical device holster comprising at least a scaffold upon which are hung in use a medical device and any associated parts, the scaffold being arranged to releasably secure the medical device within the scaffold, and to release the medical device from the scaffold for use; the bag being permanently openable along one or more major edges thereof to permit access to the medical device contained within the holster when in use.

In one example the bag is provided with a tearable seal along at least a first of the major edges thereof, to permit the bag to be torn open along the seal when the medical device contained within the bag when in use is to be deployed.

In another example the scaffold has a foldable hinge about which in use the scaffold is folded shut to secure the medical device within the scaffold, and is unfolded to release the medical device from the scaffold for use.

From another aspect there is also provided a medical device sterile barrier system, comprising: a flexible walled bag, the interior of which is sterile when in use, the bag having at least a base portion, a major front wall and major rear wall, the base portion being flat so as to permit the bag to stand upright on the base portion or sit flat on the major front or rear wall when in use to store a medical device therein, the major front wall and the major rear wall being substantially upwards pointing and vertically oriented when the bag is stood on the base portion; a medical device holster arranged in use to secure a medical device in place within the flexible walled bag; and a releasable seal provided along at least one edge of the bag to permit access to the interior of the bag to retrieve the medical device holster from within the bag when required.

In one example lateral end walls of the bag are formed by attaching material to the base and front and rear upright walls, and forming folded seams of material along the edges thereof to provide structural rigidity to the bag.

In another example the releasable seal is a single use tearable seal arranged to be torn open by the user when the medical device is to be sealed.

Further features, embodiments, and advantages of the present invention will be apparent from the appended claims.

### BRIEF DESCRIPTION OF THE DRAWINGS

Embodiments of the invention will now be described with reference to the accompanying drawings, wherein like reference numerals refer to like parts, and wherein:

FIG. 1 is perspective view of a sterile barrier packaging system according to an embodiment of the invention;

FIG. 2 is an underside view of the sterile packaging system of FIG. 1;

FIG. 3 is an overhead view of the interior of the sterile packaging system of FIG. 1;

FIG. 4 is a side view of an internal holster arrangement of the sterile packaging system of FIG. 1;

FIG. 5 is a further side view of the internal holster arrangement, showing the holster unfolded;

FIG. 6 is a diagram showing the arrangement of an internal frame within the bag of the sterile packaging system of the present embodiment;

FIG. 7 is a view of a first type of prior art medical sterile barrier packaging system;

FIG. 8 is a view of a second type of prior art medical sterile barrier packaging system;

FIG. 9 is a drawing of two types of aseptic presentation of medical instruments to a surgeon during a surgical procedure;

FIG. 10 is a collection of photographs of different prior art polymer support frames for medical instruments; and

FIG. 11 is a photograph of a prior art generic blister pack tray which can contain the support frames for medical instruments.

### OVERVIEW OF EMBODIMENTS

Embodiments of the invention provide an improved medical device sterile barrier packaging system that comprises a flexible walled bag having a base, and opposite side walls connected to the base and that fold in opposite directions upwards to meet at an apex above the base. Where the opposite side walls meet a tearable seal is formed to seal the bag, which can be torn open by user action when it is needed to access the contents. Lateral side walls are provided on the other sides of the base other than those connected to the opposite side walls, and which seal the ends of the bag. The side walls of the bag are formed from a breathable, waterproof, non-woven material such as for example Tyvek®. At least part of one wall may be formed from a clear plastic film, to allow the contents of the bag to be viewed. Because a base wall is provided the bag is self standing.

Within the bag is contained a sterile medical device, such as for example a surgical instrument. The surgical instrument may in some embodiments be an electrosurgical instrument and be accompanied by ancillary parts such as RF connection leads and lumens for suction or the supply of fluid to the surgical site. The surgical instrument and its ancillary parts are held in place within the bag by a holster arrangement comprising a cardboard or plastic scaffold that is provided in one example with a foldable hinge in the center portion, about which the scaffold can be folded in half to provide a clamshell type arrangement. The scaffold once folded provides respective holding spaces for the medical device and its ancillary equipment, with each space specifically shaped for a particular part of the medical device, or a part of ancillary equipment. As noted, the scaffold is formed of two halves, with each half being formed of respective half-parts of the various holding spaces. When the two halves of the scaffold are brought together the respective holding spaces for the device and ancillary equipment are then formed.

To use the above described scaffold structure the two halves of the structure are opened, and the medical device and the ancillary equipment are then placed in the respective half holding spaces in one half of the scaffold. The other half of the scaffold is then folded about the foldable hinge in the center of the scaffold, to brings both halves of the scaffold together, with the respective half holding spaces spatially matching, so that the two halves are then closed about the medical device and its ancillary parts, gripping them within the scaffold. The enclosed scaffold then forms the complete holster within which the medical device and its ancillary parts are hung, each in their respective holding spaces in the holster. The entire arrangement can then be picked up from one end and inserted into the bag, which can then be sterilized and sealed along the tearable seal.

When in use during a surgical procedure, the sealed bag containing the medical device can be brought into the operating theater, and the bag opened by tearing the top of the bag open along the tearable seal. The holster containing the medical device and ancillary parts is then lifted out of the bag aseptically, and laid on a sterile surface. The clamshell type scaffold is then opened by rotating one of the halves of the scaffold about the foldable hinge, thus exposing the medical device and its ancillary parts, which can then simply be lifted out of the scaffold and assembled as needed. Thus, there is no need detach the medical device or its ancillary parts from holding tabs or the like, and the device is immediately available for assembly and use once the clamshell scaffold forming the holster has been opened by folding back along the foldable hinge.

In addition to the scaffold, in some embodiments the bag is provided with an internal frame, which may be part of the holster or separate therefrom. The frame distends the bag by keeping the side walls of the bag projected outwards, away from the medical device stored in the holster formed from the clamshell scaffold. This prevents any sharpened end effector that may be present on the device (for example where the medical device is a surgical instrument) from piercing the side walls of the bag and hence compromising the sterile interior of the bag.

### DETAILED DESCRIPTION OF AN EMBODIMENT

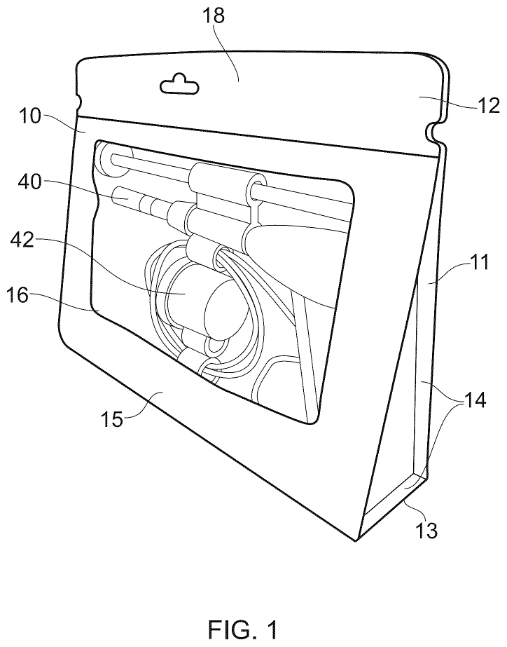

Further features of embodiments of the invention will become apparent from the following description of a specific embodiment, made with respect to FIGS. 1 to 6. In this respect FIG. 1 shows an example sterile barrier packaging system for a medical device according to the present disclosure. The system comprises a sealed bag 10, formed from a breathable, waterproof, non-woven material such as Tyvek® or the like. In this example the bag 10 is formed from a single piece of Tyvek with a window 16 formed therein from a clear, non-permeable, plastic film. A rear wall 11 extends downwards to a rear fold, where the material is then folded to form a base 13, and from the base a front fold is provided, where the material is then folded upwards to provide a front wall 15, within which the window 16 is formed. The rear wall 11 and the front wall 15 both extend upwards and meet at the top of the bag 10, where they are glued, heat-sealed (to activate adhesive), or welded together along their width to form sealed top area 18. The sealed top area 18 has notches cut into each side, defining a tearable portion 12 that extends laterally from one side of the bag to the other beneath the sealed top area 18, and along which the bag 10 may be physically torn open, thus removing the sealed top area 18 and allowing access to the sterile interior.

In other examples instead of the whole bag being formed from Tyvek® or similar non-woven breathable material, only the side walls are formed from Tyvek® or similar non-woven breathable material, and the front wall 15 and rear wall 11 are instead formed from a polymer material that is easier to tear than a material such as Tyvek®. In this case the tearable seal formed in the sealed top area 18 may be easier to form, and easier for a user to tear open.

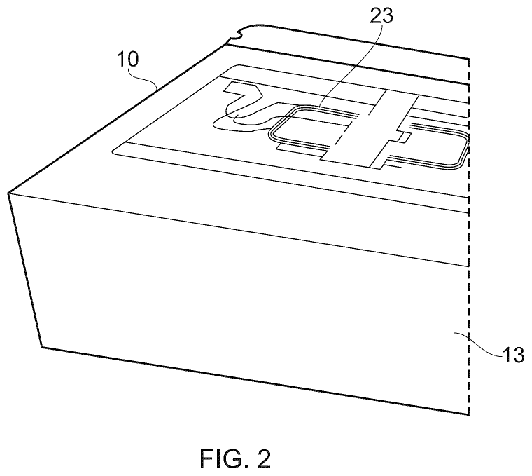

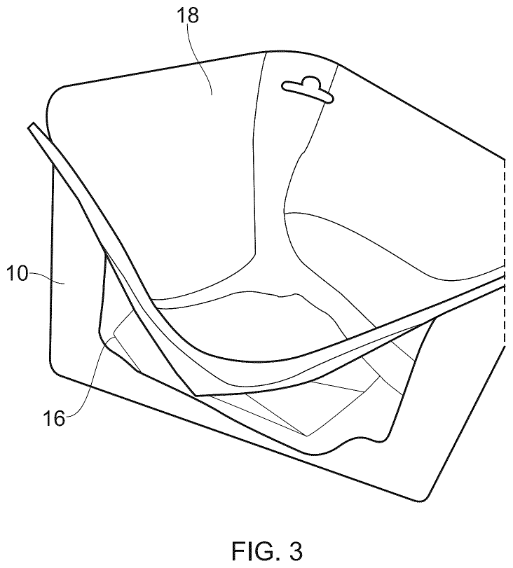

FIGS. 2 and 3 show different views of the bag 10, with FIG. 2 illustrating the base 13, and rear wall 11 of the bag 10. Printed on the rear wall is an infographic 23 illustrating the contents of the bag, and the arrangement of the medical device and ancillary parts within the holster contained within the bag. FIG. 3 illustrates the interior of the bag 10 when empty and open, and shows the top area 18 formed of glue sheets provided on the inner rear and front walls. When the glue sheets are brought together the top area 18 is then sealed shut. In other examples the bag closure may be a heat seal (to activate heat-activated adhesive), or via a weld.

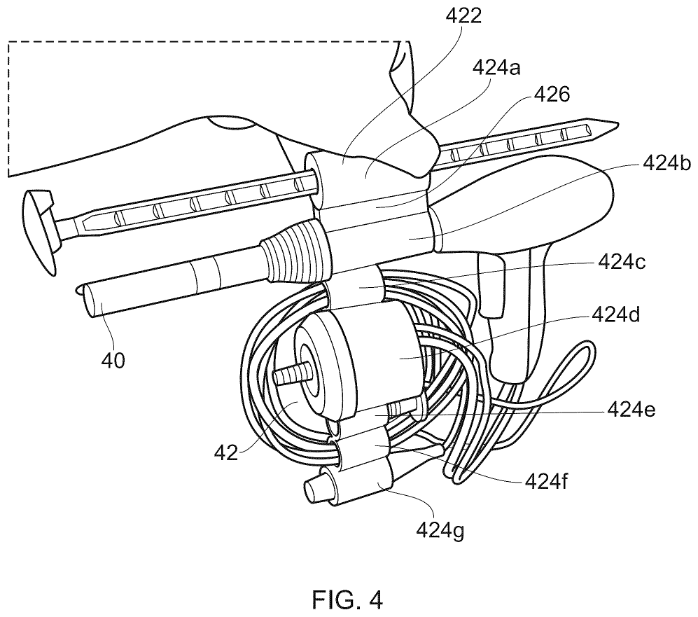

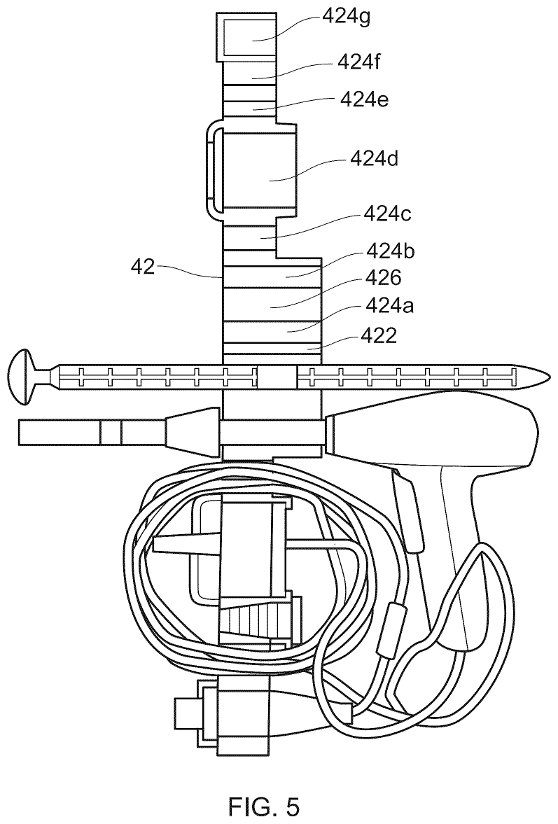

FIGS. 4 and 5 show the instrument holster 42, holding the medical device 40, which in this case is an electrosurgical morcellator instrument. FIG. 4 shows the holster 42 in the closed position, securely storing the medical device 40, whereas FIG. 5 shows the holster in the open position. As will be seen from FIG. 5 first of all, the holster 42 comprises a clamshell type scaffold that is provided with a foldable hinge portion 422 in the centre of the scaffold, and about which the scaffold may be folded in half, so as to bring each half together to provide the complete holster as shown in FIG. 4. Returning to FIG. 5, the scaffold is provided with respective device or instrument holding portions 424a to 424g, which are formed in halves in each half of the scaffold, and shaped such that when brought together when the scaffold is folded closed, they each securely hold a specific piece of the medical device 40 or ancillary equipment thereof in a respective holding portion 424a to 424g.

The scaffold 42 may be formed from cardboard, or from plastic, but an important feature of the scaffold is the amount of material that it contains. In this respect, it is sufficient for each respective holding portion 424a to 424g to be sized such they are able to securely hold each piece of the medical device 40, but do not fully enclose or encompass them. Specifically, by way of example, the scaffold holding portions 424a to 424g may be fixed such that significant portions of the parts of the medical device 40 project from each end of the holding portions 424a to 424g of the scaffold, and hence are visible to the user within the bag. That is, the scaffold forms essentially a strip or belt of material on which the medical device 40 and its ancillary parts are hung, with large majority portions of the medical device 40 projecting laterally outwards of the strip to either side. In this respect, the strip of the scaffold supports in this case significantly less than 50% of the lateral extent of the medical device, and in the example shown the amount of support of the lateral extent of the medical device may be as low as 20% of the length of the device. That is, as little as 20% of the medical device may be directly in contact with and supported by the scaffold, with the remainder of the lateral extent of the medical device extending unsupported to either side of the scaffold. In this way, material use for the scaffold is minimised whilst the support provided to the medical device within the sealed bag is not compromised.

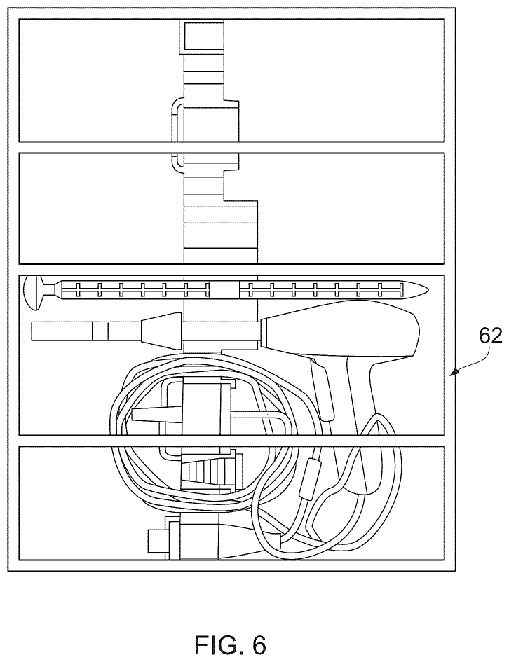

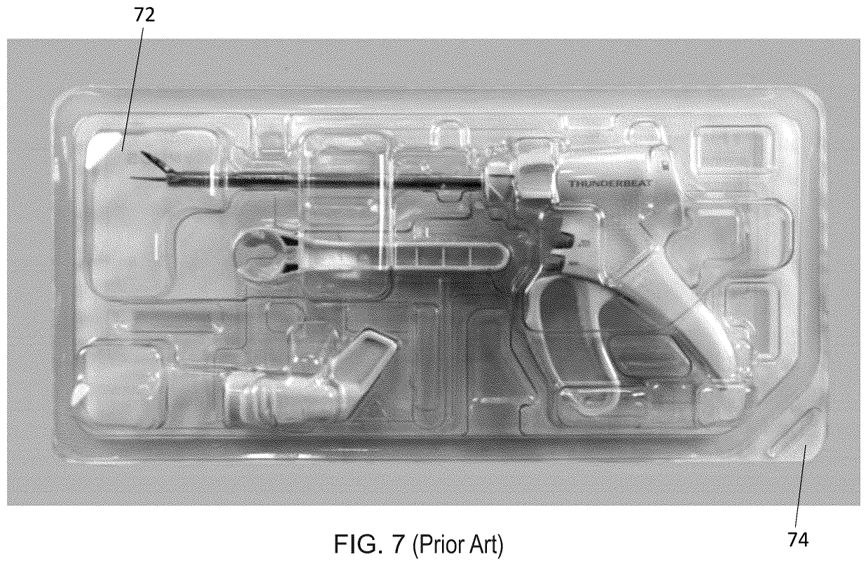

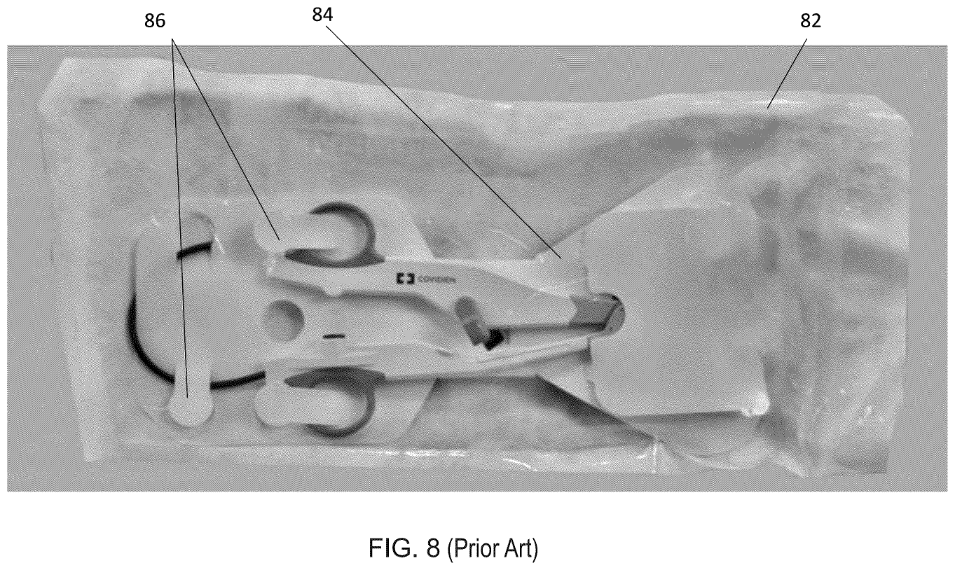

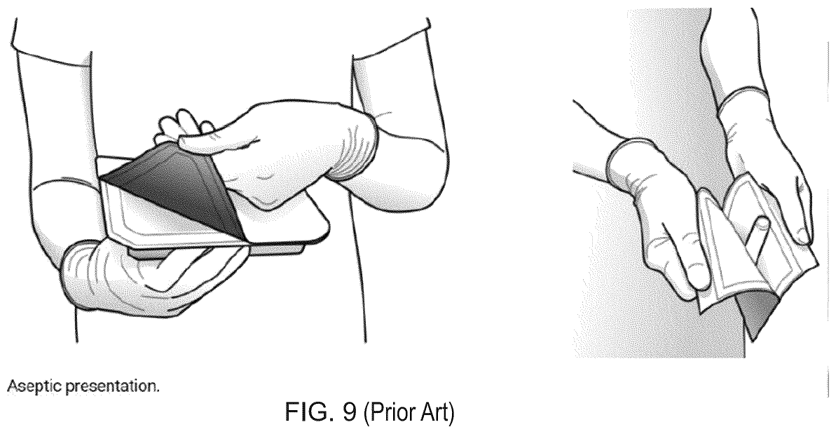

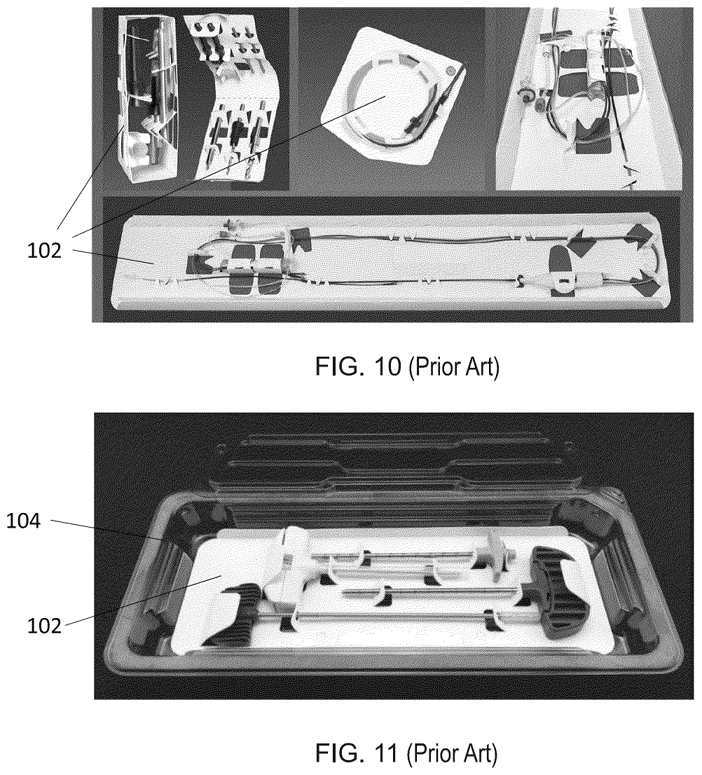

FIG. 6 shows an optional feature that may be used to provide a further embodiment. That is, within FIG. 6 an internal frame 62 is provided to distend the bag 10, and to keep the inner walls of the bag away from the medical device 40. As mentioned previously, this is to ensure that the walls of the bag are not pierced by any sharp end effector on the medical device 40, particularly where the device is a surgical instrument. The frame 62 may be part of the holster 42, or may be separate therefrom, and may be formed from cardboard or plastic.

In use, in order to store the medical device 40 within the bag 10, first of all the holster 42 is laid out on a sterile surface in the open configuration shown in FIG. 5. The medical device 40 and its ancillary parts are then arranged within ½ of the holster, by being placed within the respective holding portions 424 of the holster scaffold. Once the medical device 40 and the ancillary parts are arranged within the respective holding portions for two four of the holster scaffold, the other half of the holster scaffold is then folded about the foldable hinge 422, so as to bring the corresponding respective halves of the holding portions 424 into correspondence with the holding portions 424 into which the medical device 40 and the ancillary parts have been inserted. The two respective halves of the scaffold of the holster then form respective instrument holding portions 424a to 424g, this securing the medical device 40 and the ancillary parts within the holster 40.

Once the holster 40 has been loaded with the medical device 40 and the ancillary parts, it is then inserted into an open bag 10, such as shown in FIG. 3. Once the holster is securely within the interior of an open bag 10, the glue seal provided on the top area 18 of the bag can be closed, by bringing the front and rear walls of the bag together to seal the bag. In other examples the bag may be sealed via a heat-activated adhesive, or via a welded closure method. The bag, and the medical device inside, can then be sterilised, for example by being exposed to radiation, or via Ethylene Oxide Sterilization. Because of the nature of the bag as being breathable but waterproof, the medical device inside the bag will then stay sterilised until the bag is opened, within a surgical procedure. Where the bag is formed completely from a non-breathable polymer with no breathable membranes forming any walls thereof, then sterilization occurs via radiation.

In order to use the medical device 40 within a surgical procedure, the sealed bag 10 containing the medical device 40 is brought into the operating theatre and opened by a theatre assistant then grasping the top area 18 of the bag and tearing the area off via the tearable portion 12, thus opening the bag. The contents of the bag are then aseptically presented to the surgeon or his assistant. The surgeon or scrub nurse assistant then reaches inside the bag and removes the holster carrying the medical device 40 and places the holster on a sterile surface. The holster is then opened by folding back half of the holster scaffold about the central foldable hinge, thus exposing all of the medical device 40 and the ancillary equipment, which can then be lifted from the scaffold of the holster. Thus, all that is required is for the surgeon or scrub nurse assistant to remove the loaded holster from the bag, and then to open the holster by folding back half of the scaffold, whereupon the medical device 40 and its ancillary instruments are immediately available and accessible. There is thus no need to remove the device and ancillary parts from a paper scaffold by unpicking individually securing tabs, as is common in the prior art. Instead, all of the device and the ancillary parts are immediately accessible at the same time once the holster has been opened by folding back the holster scaffold.

Various modifications may be made to the above to provide further embodiments. For example, we have described above a bag of rectangular base with a substantially triangular cross section formed from bring the major front and rear walls together. However, this arrangement is not essential and other shaped bags can be formed, for example with rectangular cross-sections, or different shaped bases.

Moreover, the holster 42 can be adapted to have greater or fewer respective holding portions 424, and of different shape, depending on the medical instrument to be contained within the bag, it being understood that the precise holster design is dependent on the device 40 to be stored, within the general constraints described above. The described holster arrangement is particularly effective for elongated instruments, that can be broken down and stored in parts in the holster arrangement. In this respect, the prior art arrangements are often not effective for long instruments, as it can be hard to seal a Tyvek lid onto a thermoplastic tray over the length of a long instrument i.e. typically longer than 30 cm. Even if the instrument cannot be broken down, providing a long bag according to the present disclosure will be easier to seal than the prior art tray arrangements. Moreover, the use of the described holster arrangement also prevents the use of lots of wasted plastic material that would be generated to produce the tray 72 of the prior art for an elongated instrument.

With respect to the form of the medical device 40, it may be any medical device that it is desired to keep sterile, and particularly any surgical instrument, and in particular an electrosurgical instrument, such as an RF shaver, RF morcellator, or RF forceps device.

Various modifications, whether by way of addition, deletion, or substitution may be made to above mentioned embodiments to provide further embodiments, any and all of which are intended to be encompassed by the appended claims.

For example, in a further embodiment the scaffold may be of a different design more akin to the prior art, where there is a fixed (i.e. non foldable or movable) template within which the medical device or accessories are threaded through. Whilst such does not have the quick release functionality of the hinged arrangement described above, the other advantages of the releasably securable bag in terms of storability and usage of materials are still retained with such an arrangement.

In a yet further embodiment the holster and the medical device may be kept within a second, sealable, non-woven, waterproof bag that is within the first bag, such that the first bag is then an outer bag to the second, inner bag. This has advantages that then the respective seals on each bag can be made slightly weaker, thus making the seals easier to release in use.

1. A medical device sterile barrier system, comprising:
a flexible walled bag, the interior of which is sterile when in use; and
a medical device holster contained within the bag, the medical device holster comprising at least a scaffold upon which are hung in use a medical device and any associated parts, the scaffold being arranged to releasably secure the medical device within the scaffold, and to release the medical device from the scaffold for use;
the bag being permanently openable along one or more major edges thereof to permit access to the medical device contained within the holster when in use.

2. A system according to claim 1, wherein the bag is provided with a tearable seal along at least a first of the major edges thereof, to permit the bag to be torn open along the seal when the medical device contained within the bag when in use is to be deployed.

3. A system according to claim 1, wherein the scaffold has a foldable hinge about which in use the scaffold is folded shut to secure the medical device within the scaffold, and is unfolded to release the medical device from the scaffold for use.

4. A system according to claim 3, wherein the scaffold of the medical device holster comprises a first half and a second half arranged about the foldable hinge, wherein in use the first half and the second half are brought together in alignment by folding about the foldable hinge to from at least one clamshell-type holding portion within which the medical device is secured therein.

5. A system according to claim 4, wherein respective clamshell-type holding portions are formed by the first half and the second half when brought together in alignment to secure the medical device and any associated parts separately in the respective clamshell-type holding portions.

6. A system according to claim 5, wherein the clamshell-type holding portions are sized such that the medical device extends laterally on either side thereof, the clamshell portions being arranged such that they secure a middle region of the device whereby the medical device hangs therefrom.

7. A system according to claim 6, wherein the clamshell-type holding portion supports less than 50% of the lateral extension of the medical device.

8. A system according to claim 7, wherein the clamshell-type holding portion supports less than 30% of the lateral extension of the medical device.

9. A system according claim 1, wherein the bag is formed from either:
i) one or more non-breathable polymer materials and no other breathable materials; or
ii) a combination of one or more breathable, water-resistant materials and one or more non-breathable polymer materials.

10. A system according to claim 1, wherein the bag is formed from a folded piece of flexible material folded to form a first front upright wall, a flat base portion, and a second rear upright wall, the first and second upright walls being secured together at the upper major edge thereof to releasably seal the bag.

11. A system according to claim 10, and further comprising lateral end walls of the bag formed by attaching material to the base and front and rear upright walls, and forming folded seams of material along the edges thereof to provide structural rigidity to the bag.

12. A system according to claim 10, wherein when the bag is sealed the bag is of substantially triangular cross section when viewed along a laterally extending axis of the bag.

13. A system according to claim 1, and further comprising an internal frame to distend the bag, whereby to keep the inner walls of the bag away from the medical device.

14. A medical device sterile barrier system, comprising:
a flexible walled bag, the interior of which is sterile when in use, the bag having at least a base portion, a major front wall and major rear wall, the base portion being flat so as to permit the bag to stand upright on the base portion or sit flat on the major front or rear wall when in use to store a medical device therein, the major front wall and the major rear wall being substantially upwards pointing and vertically oriented when the bag is stood on the base portion;
a medical device holster arranged in use to secure a medical device in place within the flexible walled bag; and
a releasable seal provided along at least one edge of the bag to permit access to the interior of the bag to retrieve the medical device holster from within the bag when required.

15. A system according to claim 14, and further comprising lateral end walls of the bag formed by attaching material to the base and front and rear upright walls, and forming folded seams of material along the edges thereof to provide structural rigidity to the bag.

16. A system according to claim 14, wherein the releasable seal is a single use tearable seal arranged to be torn open by the user when the medical device is to be sealed.

17. A system according to claim 14, wherein the medical device holster opens and closes about a foldable hinge to receive the medical device within the holster and secure it therein, and to permit subsequent access to the medical device.

18. A system according to claim 1, and further comprising a secondary sealable bag contained within the flexible walled bag, within which in use the medical device holster containing the medical device is stored.

19. A method of assembling a medical device sterile barrier system, the medical device sterile barrier system comprising:
a flexible walled bag, the interior of which is sterile when in use; and
a medical device holster contained within the bag, the medical device holster comprising at least a scaffold upon which are hung in use a medical device and any associated parts, the scaffold being arranged to releasably secure the medical device within the scaffold, and to release the medical device from the scaffold for use;
the bag being permanently openable along one or more major edges thereof to permit access to the medical device contained within the holster when in use;
the method comprising:
opening the medical device holster;
arranging the medical device and any associated parts within respective holder portions of the holster;
closing the holster to secure the medical device within the holster;
inserting the holster within the bag;
sealing the bag; and
sterilizing the bag using any one or more of exposure to sterilizing gas or sterilizing radiation.

20. A method of deploying a medical device sterile barrier system, the medical device sterile barrier system comprising:
a flexible walled bag, the interior of which is sterile when in use; and
a medical device holster contained within the bag, the medical device holster comprising at least a scaffold upon which are hung in use a medical device and any associated parts, the scaffold being arranged to releasably secure the medical device within the scaffold, and to release the medical device from the scaffold for use;
the bag being permanently openable along one or more major edges thereof to permit access to the medical device contained within the holster when in use;
the method comprising the steps:
receiving the system into a sterile area of an operating theatre;
tearing open the seal of the system;
removing the medical device holster bearing the medical device from the bag;
opening the holster to release the medical device from the holster; and
retrieving the medical device directly from within the holster by picking the device up from the holster;
wherein the opening of the holster renders the medical device immediately available to be picked up from the holster without a user having to release any further device retaining mechanisms.
